(19) United States
(12) Patent Application Publication
(10) Pub. No.: US 2013/0035650 A1
(43) Pub. Date: Feb. 7, 2013

(54) DEAD SKIN REMOVING DEVICE AND METHOD FOR FORMING THE SAME

(71) Applicant: Wei-Jen Wang, Taichung City (TW)

(72) Inventor: Wei-Jen Wang, Taichung City (TW)

(21) Appl. No.: 13/558,920

(22) Filed: Jul. 26, 2012

(30) Foreign Application Priority Data
Aug. 1, 2011 (TW) 100214179

(51) Int. Cl. A61M 35/00; B32B 37/06

(52) U.S. Cl. 604/292; 156/60

(57) ABSTRACT

A dead skin removing device for a user's hand includes a liquidproof plastic glove which defines an inner space and which includes a palm piece and a back piece that are configured to be matingly plastic-welded to each other, and a patch member which is bonded to a portion of the palm piece or the back piece. The patch member is soaked with an exfoliating agent such that once the user's hand is inserted into the liquidproof plastic glove, a region of the user's hand is brought into intimate contact with the exfoliating agent. A method of forming the dead skin removing device is also disclosed.

### CROSS-REFERENCE TO RELATED APPLICATION

This application claims priority of Taiwanese application no. 100214179, filed on Aug. 1, 2011.

### BACKGROUND OF THE INVENTION

1. Field of the Invention

This invention relates to a dead skin removing device, more particularly to a dead skin removing device for a user's hand, and a method for forming the same.

2. Description of the Related Art

There are many methods to remove excess layers of dead skin cells from the surface of skin to give the skin a shiny and youthful look, such as mechanical scrubbing, laser treatment, chemical peeling treatment, etc. However, mechanical scrubbing will facilitate build up of the dead skin cells since skin has a natural protection mechanism to increase its thickness for preventing skin injury, whereas laser treatment is costly, and chemical peeling is time-consuming. When chemical peeling treatment is applied to a user's hand, a glove made of non-woven fabric and soaked with an exfoliating agent may be used. When wearing such a glove, the user cannot perform household chores, such as cleaning, since the glove is not liquidproof.

### SUMMARY OF THE INVENTION

Therefore, an object of the present invention is to provide a dead skin removing device for a user's hand.

Another object of the present invention is to provide a method of forming a dead skin removing device for a user's hand. A user using the dead skin removing device to remove dead skin on his/her hand can still do household chores with dexterity.

According to the first aspect of this invention, a dead skin removing device for a user's hand comprises :

a liquidproof plastic glove which defines an inner space for receiving the user's hand and which includes a palm piece and a back piece each being formed in a shape of a human hand, the palm piece having a first thumb portion, a first finger portion, and a palm portion, the back piece having a second thumb portion, a second finger portion, and a back portion which are configured to be matingly plastic-welded with the first thumb portion, the first finger portion, and the palm portion, respectively, such that the inner space is formed between the palm piece and the back piece, and such that bottom margins of the palm portion and the back portion cooperatively define an opening to permit insertion of the user's hand into the inner space therethrough; and

a patch member which is bonded to one of the first and second thumb portions, the first and second finger portions, the palm portion, and the back portion in the inner space, and which is soaked with an exfoliating agent such that the patch member intimately contacts a region of the user's hand to be treated by the exfoliating agent once the user's hand is inserted into the inner space through the opening.

According to another aspect of this invention, a method of forming a dead skin removing device for a user's hand, comprising the steps of:

(a) providing a palm piece and a back piece each being formed in a shape of a human hand, the palm piece having a first thumb portion, a first finger portion, and a palm portion, the back piece having a second thumb portion, a second finger portion, and a back portion which are configured to mate with the first thumb portion, the first finger portion, and the palm portion, respectively;

(b) providing a patch member including front and back layers made of substantially the same material;

(c) bonding the back layer of the patch member to one of the first and second thumb portions, the first and second finger portions, the palm portion, and the back portion;

(d) soaking the front layer of the patch member with an exfoliating agent;

(e) joining the palm and back pieces by plastic-welding, including the sub-steps of: (e1) plastic-welding a marginal region of the first thumb portion to a marginal region of the second thumb portion, (e2) plastic-welding a marginal region of the first finger portion to a marginal region of the second finger portion, and (e3) plastic-welding a marginal region of the palm portion to a marginal region of the back portion, such that an inner space for receiving the user's hand is formed between the palm piece and the back piece, such that bottom margins of the palm portion and the back portion are left unwelded to provide an opening for insertion of the user's hand into the inner space therethrough, and such that the patch member intimately contacts a region of the user's hand to be treated by the exfoliating agent once the user's hand is inserted into the inner space through the opening.

### BRIEF DESCRIPTION OF THE DRAWINGS

Other features and advantages of the present invention will become apparent in the following detailed description of the preferred embodiments of the invention, with reference to the accompanying drawings, in which:

FIG. 1 is a schematic view of the preferred embodiment of a dead skin removing device according to the present invention;

FIG. 2 is a partially sectioned view taken along line II-II shown in FIG. 1;

FIG. 3 is similar to FIG. 1 but illustrating that a user's hand is inserted into the dead skin removing device;

FIG. 4 is a schematic view of a modified embodiment of a dead skin removing device according to the present invention; and

FIG. 5 is a schematic view of another modified embodiment of a dead skin removing device according to the present invention.

### DETAILED DESCRIPTION OF THE PREFERRED EMBODIMENTS

Before the present invention is described in greater detail, it should be noted herein that like elements are denoted by the same reference numerals throughout the disclosure.

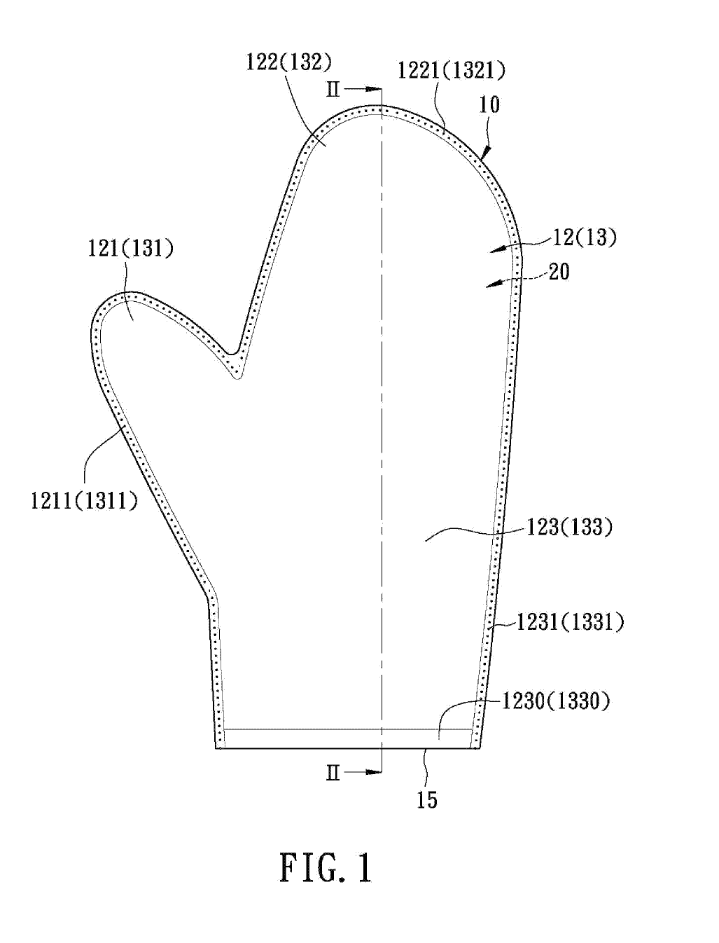

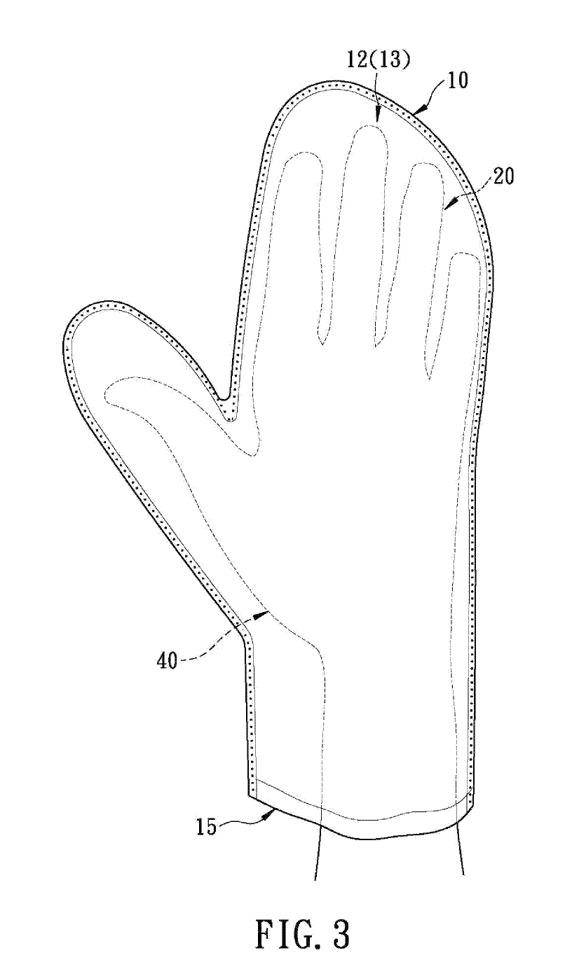

FIGS. 1 to 3 illustrate the preferred embodiment of a dead skin removing device for a user's hand 40 according to this invention. The dead skin removing device includes a liquidproof plastic glove 10 and a patch member 20. A method for forming the dead skin removing device comprises the following steps (a) to (e).

In step (a), palm and back pieces 12, 13 of the liquidproof plastic glove 10 are provided, each of which is formed in a shape of a human hand, and each of which is made of liquidproof plastic, such as materials commonly used for forming a plastic hand glove. The palm piece 12 has a first thumb portion 121, a first finger portion 122, and a palm portion 123. The back piece 13 has a second thumb portion 131, a second finger portion 132, and a back portion 133 which are configured to mate with the first thumb portion 121, the first finger portion 122, and the palm portion 123, respectively.

Instep (b), the patch member 20 is provided. The patch member 20 has a back layer 21 and a front layer 22 that are made of substantially the same liquid-adsorbable material. In this embodiment, the back layer 21 and the front layer 22 are formed in one-piece and are made of nonwoven fabric.

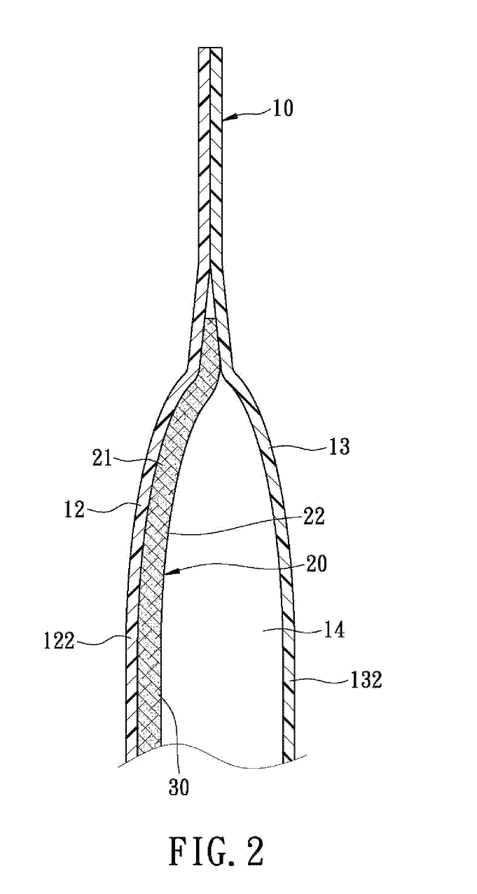

As best shown in FIG. 2, in step (c), the back layer 21 of the patch member 20 is adhesively bonded to one of the first and second thumb portions 121, 131, the first and second finger portions 122, 132, the palm portion 123, and the back portion 133 (see also FIG. 1). In step (d), the front layer 22 of the patch member 20 is soaked with an exfoliating agent 30. The exfoliating agent 30 may be any conventional exfoliating agents.

The patch member 20 can be bonded to the palm piece 12 and/or the back piece 13 by many possible methods. For example, the patch member 20 may have an adhesive layer (not shown) formed on the back layer 21 and covered by a release film (not shown), and may be bonded to the palm piece 12 and/or the back piece 13 by virtue of the adhesive layer after removal of the release film. On the other hand, the patch member 20 and the palm piece 12 (or the back piece 13) may be passed through a heating device (such as a heating roller, not shown) such that the palm piece 12 (or the back piece 13) is melt-bonded to the patch member 20. When the adhesive layer is used in step (c), step (c) can be implemented before or after step (d). When the melt-bonding method is used in step (c), step (c) is preferably implemented before step (d).

Step (e) is a step for plastic welding the palm piece 12 to the back piece 13, and includes the sub-steps of: (e1) plastic-welding a marginal region 1211 of the first thumb portion 121 to a marginal region 1311 of the second thumb portion 131, (e2) plastic-welding a marginal region 1221 of the first finger portion 122 to a marginal region 1321 of the second finger portion 132, and (e3) plastic-welding a marginal region 1231 of the palm portion 123 to a marginal region 1331 of the back portion 133, such that an inner space 14 for receiving the user's hand is formed between the palm piece 12 and the back piece 13, such that bottom margins 1230, 1330 of the palm portion 123 and the back portion 133 are left unwelded to provide an opening 15 for insertion of the user's hand 40 into the inner space 14 therethrough, and such that the patch member 20 intimately contacts a region of the user's hand 40 to be treated by the exfoliating agent 30 once the user's hand 40 is inserted into the inner space 14 through the opening 15. Accordingly, the dead skin removing device of this invention is obtained, in which the patch member 20 is disposed in the inner space 14 defined by the liquidproof plastic glove 10.

As shown in FIGS. 1 and 2, in this embodiment, the patch member 20 is bonded to an entire area of the palm piece 12, and thus, the patch member 20 intimately contacts the palm side of the user's hand 40 once the user's hand 40 is inserted into the liquidproof plastic glove 10. Of course, the dead skin removing device may have two or more patch members 20 respectively bonded to two or more portions of the palm and back pieces 12, 13.

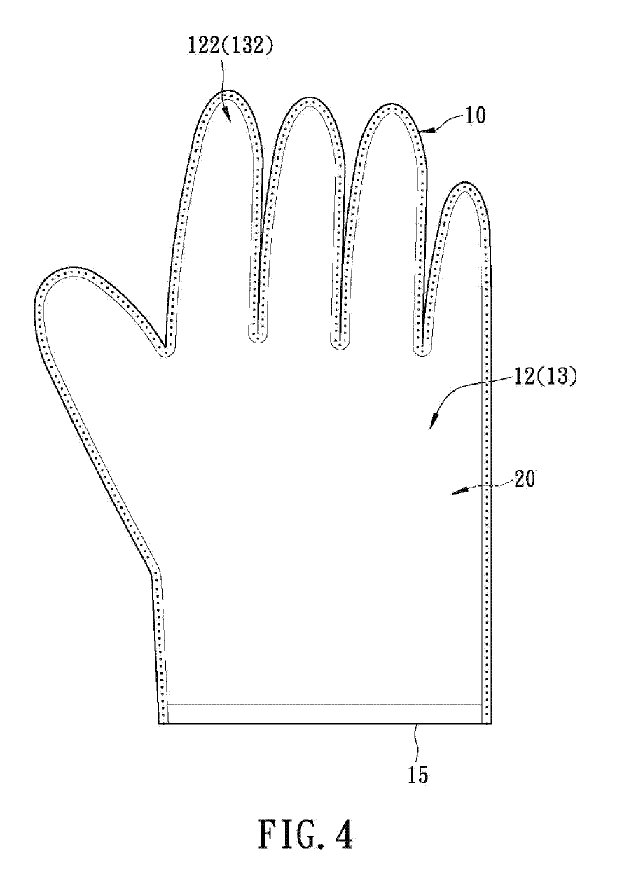

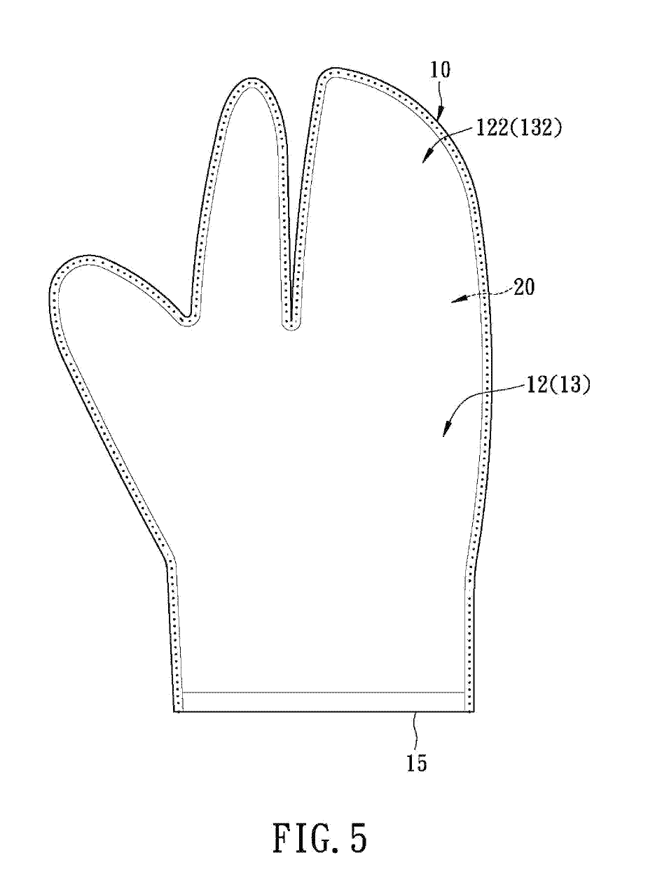

In the embodiment as shown in FIG. 1, the liquidproof plastic glove 10 is formed into a mitten-type hand glove. In other preferred embodiments, the liquidproof plastic glove 10 can be formed into a five-finger type hand glove (see FIG. 4) or a three-finger type hand glove (see FIG. 5) by varying the shapes of the first and second finger portions 122, 132.

Preferably, step (e) is implemented by heat-sealing or ultrasonic sealing the marginal regions 1211, 1221, 1231 of the first thumb portion 121, the first finger portion 122, and the palm portion 123 respectively to the marginal regions 1311, 1321, 1331 of the second thumb portion 131, the second finger portion 132, and the back portion 133.

In this embodiment, because the patch member 20 is bonded to an entire area of the palm piece 12, and thus, step (e) is implemented after steps (c) and (d).

In other modified embodiment, especially when the adhesive layer is used to bond the back layer 21 of the patch member 20 to the palm piece 12 or the back piece 13 in step (c), and when the back layer 21 of the patch member 21 is not bonded to an entire area of the palm piece 12 or the back piece 13, the sequence of step (c) and sub-steps (e1) to (e3) may be varied as described below.

In the event that the back layer 21 of the patch member 20 is bonded to one of the first and second thumb portions 121, 131 in step (c), sub-step (e1) is implemented after step (c), and sub-steps (e2) and (e3) can be implemented before or after step (c).

In the event that the back layer 21 of the patch member 20 is bonded to one of the first and second finger portions 122, 132 in step (c), sub-step (e2) is implemented after step (c), and sub-steps (e1) and (e3) can be implemented before or after step (c).

In the event that the back layer 21 of the patch member 20 is bonded to one of the palm portion 123 and the back portion 133 in step (c), sub-step (e3) is implemented after step (c), and sub-steps (e1) and (e2) can be implemented before or after step (c).

The dead skin removing device may be worn by the user preferably for a predetermined period of time of about 30 to 120 minutes, depending on the components of the exfoliating agent 30, and then taken off. Dead skin on the user's hand 40 will naturally and gradually slough off 2-3 days later.

In the present invention, because the plastic glove 10 of the dead skin removing device is liquidproof, the user can still do household chores while his/her hand is undergoing treatment by the exfoliating agent 30, and his/her hand dexterity will not be significantly affected. Besides, the size of the patch member 20 can be varied based on actual requirements. For example, since the back of the user's hand 40 is less likely to have dead skin, such as callus, the patch member 20 may be selectively bonded only to one of the palm and back pieces 12, 13 to treat the palm side of the user's hand 40, so that the amount of the exfoliating agent 30 may be reduced compared to conventional gloves used for chemical peeling treatment.

While the present invention has been described in connection with what are considered the most practical and preferred embodiments, it is understood that this invention is not limited to the disclosed embodiments but is intended to cover various arrangements included within the spirit and scope of the broadest interpretations and equivalent arrangements.

What is claimed is:

1. A dead skin removing device for a user's hand, comprising:
a liquidproof plastic glove which defines an inner space for receiving the user's hand and which includes a palm piece and a back piece each being formed in a shape of a human hand, said palm piece having a first thumb portion, a first finger portion, and a palm portion, said back piece having a second thumb portion, a second finger portion, and a back portion which are configured to be matingly plastic-welded with said first thumb portion, said first finger portion, and said palm portion, respectively, such that said inner space is formed between said palm piece and said back piece, and such that bottom margins of said palm portion and said back portion cooperatively define an opening to permit insertion of the user's hand into said inner space therethrough; and
a patch member which is bonded to one of said first and second thumb portions, said first and second finger portions, said palm portion, and said back portion in said inner space, and which is soaked with an exfoliating agent such that said patch member intimately contacts a region of the user's hand to be treated by said exfoliating agent once the user's hand is inserted into said inner space through said opening.

2. The dead skin removing device of claim 1, wherein said patch member is bonded to an entire area of said palm piece.

3. A method of forming a dead skin removing device for a user's hand, comprising the steps of:
(a) providing a palm piece and a back piece each being formed in a shape of a human hand, the palm piece having a first thumb portion, a first finger portion, and a palm portion, the back piece having a second thumb portion, a second finger portion, and a back portion which are configured to mate with the first thumb portion, the first finger portion, and the palm portion, respectively;
(b) providing a patch member including front and back layers made of substantially the same material;
(c) bonding the back layer of the patch member to one of the first and second thumb portions, the first and second finger portions, the palm portion, and the back portion;
(d) soaking the front layer of the patch member with an exfoliating agent;
(e) joining the palm and back pieces by plastic-welding, including the sub-steps of: (e1) plastic-welding a marginal region of the first thumb portion to a marginal region of the second thumb portion, (e2) plastic-welding a marginal region of the first finger portion to a marginal region of the second finger portion, and (e3) plastic-welding a marginal region of the palm portion to a marginal region of the back portion, such that an inner space for receiving the user's hand is formed between the palm piece and the back piece, such that bottom margins of the palm portion and the back portion are left unwelded to provide an opening for insertion of the user's hand into the inner space therethrough, and such that the patch member intimately contacts a region of the user's hand to be treated by the exfoliating agent once the user's hand is inserted into the inner space through the opening.

4. The method of claim 3, wherein step (e) is implemented after steps (b) to (d).

5. The method of claim 3, wherein step (c) is implemented before or after step (d).

6. The method of claim 5, wherein, in the event that the back layer of the patch member is bonded to one of the first and second thumb portions in step (c), sub-step (e1) is implemented after step (c), and sub-steps (e2) and (e3) are implemented before or after step (c).

7. The method of claim 5, wherein, in the event that the back layer of the patch member is bonded to one of the first and second finger portions in step (c), sub-step (e2) is implemented after step (c), and sub-steps (e1) and (e3) are implemented before or after step (c).

8. The method of claim 5, wherein, in the event that the back layer of the patch member is bonded to one of the palm and the back portions in step (c), sub-step (e3) is implemented after step (c), and sub-steps (e1) and (e2) are implemented before or after step (c).

9. The method of claim 3, wherein the back layer of the patch member is adhesively bonded to an entire area of the palm piece.
